(19) United States
(12) Patent Application Publication
(10) Pub. No.: US 2017/0293730 A1
(43) Pub. Date: Oct. 12, 2017

(54) REMOTE CONTROLLED MEDICAL ASSISTANCE DEVICE

(71) Applicant: 19LABS INC., Menlo Park, CA (US)

(72) Inventors: Ram Adva FISH, Menlo Park, CA (US); Gerald Charles HOREL, British Columbia (CA)

(21) Appl. No.: 15/480,438

(22) Filed: Apr. 6, 2017

Related U.S. Application Data
(60) Provisional application No. 62/320,190, filed on Apr. 8, 2016.

(51) Int. Cl. G06F 19/00; G08C 17/02; G09B 19/00; A61B 1/227; A61B 5/145; A61B 5/0205; A61B 7/02; H04M 1/725; A61B 5/00

(52) U.S. Cl. CPC G06F 19/3418; H04M 1/72583; G08C 17/02; G06F 19/321; G09B 19/00; A61B 5/742; A61B 5/0022; A61B 5/14542; A61B 5/02055; A61B 7/02; A61B 1/227; H04W 4/22

(57) ABSTRACT

A system for treating a patient, including a computerized device with a display and a communication interface for communicating with remote mobile devices, one or more sensors for measuring medical parameters of a user, wherein the sensors are controlled by the computerized device and transfer recorded measurements to the computerized device, an application executed on the computerized device; wherein the application allows, a remote practitioner with a remote mobile device to communicate with the computerized device to control the sensors.

### RELATED APPLICATIONS

This application claims priority under 35 U.S.C. 120 from U.S. provisional application No. 62/320,190 dated Apr. 8, 2016, the disclosure of which is incorporated herein by reference.

### FIELD OF THE DISCLOSURE

The present disclosure relates generally to a device for providing medical assistance that can be controlled remotely by a medical practitioner.

### BACKGROUND OF THE DISCLOSURE

In many cases people are in need of medical assistance yet only unskilled people are available at the location to provide the assistance. Typically, businesses and homeowners have first aid kits available in case of need, based on the assumption that somebody at the location will have basic knowledge to use the contents of the first aid kit.

In many cases the available people may have little or no knowledge as to how to assist the person in need of medical assistance. Likewise the available people may not even know who to call to receive help.

It is thus desirable to have a first aid system, which can provide guidance to an available person and/or provide sensors for assessing the state of the person in need of medical assistance. Additionally, it would be helpful if a remote practitioner could use the system from a remote location, to provide guidance In deploying the sensors and or using them.

### SUMMARY OF THE DISCLOSURE

An aspect of an embodiment of the disclosure, relates to a system and method for treating a patient by a remote medical practitioner. The system includes a computerized device (e.g. a tablet computer) with various connectable medical, sensors for evaluating a patient's status and/or treating the patient. The system further includes an application that allows the user to communicate with a remote medical practitioner via a mobile device of the practitioner. The practitioner can discuss the status of the patient with the patient or with a user helping the patient and instruct the user or patient to deploy one or more sensors for examining the patient. The practitioner can then control the sensors (e.g. activate, deactivate, view measurements) via his or her mobile device. Optionally, the practitioner may control the sensors by typing text commands and/or by verbally pronouncing specific commands.

Additionally, the practitioner may explain to the user what to do and/or control the display of the computerized device, for example open menu's and/or help screens that are related to the medical problem that the user is dealing with. In an exemplary embodiment of the disclosure, the computerized device may include cameras, speakers and microphones to allow the practitioner to speak lace to face with the user. Optionally, the user can focus a camera on the patient, for example to show the practitioner a wound or to view the status of the patient. The practitioner may be able to remotely activate a distress call to emergency services to come to the location of the computerized device, for example if the patient is unable to call them directly.

There is thus provided according to an exemplary embodiment of the disclosure, a system for treating a patient, comprising:
A computerized device comprising a display and a communication interface for communicating with remote mobile devices;
One or more sensors for measuring medical parameters of a user: wherein the sensors are controlled by the computerized device and transfer recorded measurements to the computerized device;
An application executed on the computerized device; wherein the application allows a remote practitioner with a remote mobile device to communicate with the computerized device to control the sensors.
In an exemplary embodiment of the disclosure, the application accepts verbal audio commands from the remote practitioner to control the sensors. Optionally, the application accepts text commands from the remote practitioner to control the sensors. In an exemplary embodiment of the disclosure, at least one sensor communicates with the computerized device via a USB connection. Optionally, at least one sensor communicates with the computerized device via a direct local wireless connection. In an exemplary embodiment of the disclosure, the application allows the remote practitioner to control the display of menus and help guides on the display of the computerized device. Optionally, the application allows the remote practitioner to provide verbal messages that are displayed as text on the display of the computerized device. In an exemplary embodiment of the disclosure, the application accepts images from a camera and transmits them to the remote practitioner to view. Optionally, the one or more sensors are selected from the group consisting of a pulse oximeter, a blood pressure sensor, a stethoscope, an otoscope and a thermometer. In an exemplary embodiment of the disclosure, the application transmits the readings of the sensors to the remote practitioner to view. Optionally, the computerized device is a tablet. In an exemplary embodiment of the disclosure, the application allows a remote practitioner to initialize a call for help from emergency services by the computerized device.

There is further provided according to an exemplary embodiment of the disclosure, a method of treating a patient by a medical practitioner from a remote location, comprising:
Activating a computerized device comprising a display and a communication interlace for communicating with remote mobile devices;
Deploying one or more sensors for measuring medical parameters of a user; wherein the sensors communicate and receive instructions from the computerized device;
Initializing an application executed on the computerized device;
Forming communication between the application and a remote mobile device used by a remote practitioner;
Enabling the remote practitioner to control the sensors remotely by the computerized device.

In an exemplary embodiment of the disclosure, the application accepts verbal audio commands from the remote practitioner to control the sensors. Optionally, the application accepts text commands from the remote practitioner to control the sensors. In an exemplary embodiment of the disclosure, the application allows the remote practitioner to control the display of menus and help guides on the display of the computerized device. Optionally, the application allows the remote practitioner to provide verbal audio messages that are displayed as text on the display of the computerized device. In an exemplary embodiment of the disclosure, the application accepts images from a camera and transmits them to the remote practitioner to view. Optionally, the application transmits the readings of the sensors to the remote practitioner to view.

There is further provided according to an exemplary embodiment of the disclosure, a non-transitory memory device storing an executable program that can be executed on a computerized device to perform the method described above.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a system for receiving remote assistance from a medical practitioner, according to an exemplary embodiment of the disclosure;

FIG. 2 is a schematic block diagram of elements of an application for communicating with a medical practitioner, according to an exemplary embodiment of the disclosure; and

FIG. 3 is a schematic illustration of an exemplary display of a remote mobile device for communicating with a medical assistance application, according to an exemplary embodiment of the disclosure.

### DETAILED DESCRIPTION

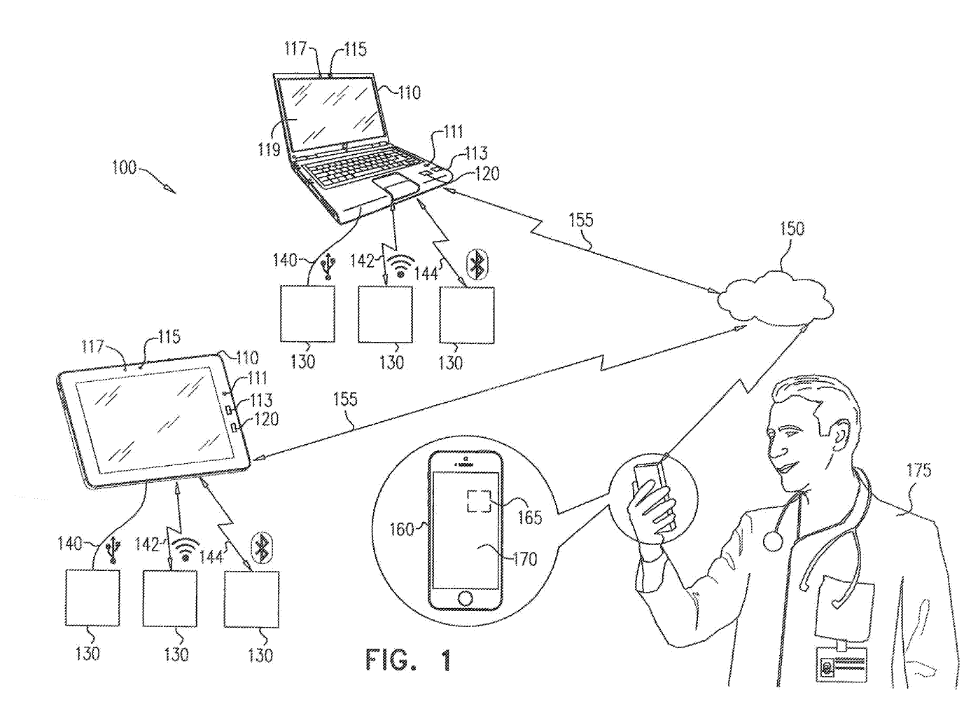

FIG. 1 is a schematic illustration of a system 100 for receiving remote assistance from a medical practitioner 175, according to an exemplary embodiment of the disclosure. Optionally, system 100 can be used by a layman in the vicinity of a patient to communicate with medical practitioner 175 and receive instructions or have the practitioner remotely control activities in system 100.

In an exemplary embodiment of the disclosure, system 100 is a medical kit including a general purpose computer 110. Optionally, the general purpose computer may be provided as a desktop computer, a laptop computer (e.g. as shown in FIG. 1), a tablet computer (e.g. as shown in FIG. 1), a smartphone or other type of computational device having a processor and memory with the capability of executing applications. In an exemplary embodiment of the disclosure, a medical assistance application 120 is stored and executed by computer 110 to provide instructions to a user, receive measurements from medical sensors 130, send information to the medical practitioner 175, receive instructions from the medical practitioner 175 and/or be controlled remotely by the medical practitioner 175.

In an exemplary embodiment of the disclosure, system 100 is provided with medical sensors 130 for use in a medical event to measure medical information from a patient, for example the sensors may include:
1. A pulse oximeter to monitor a person's oxygen saturation (SO₂);
2. A blood pressure sensor to measure a person's blood pressure;
3. A stethoscope to listen to or record a person's lungs and/or heart or other internal sounds;
4. An otoscope or similar device to view a person's ears or mouth;
5. A thermometer;
6. Other medical sensors.

Optionally, the sensors are all connectable to computer 160 and configured to transfer their measurements to the computer for providing them remotely to the medical practitioner 175 to view. In an exemplary embodiment of the disclosure, the sensors 130 may be connectable to computer 110 via a USB connection 140, a local Wi-Fi connection 142, A Blue-Tooth connection 144 or other type of local connection. Optionally, application 120 is configured to control the sensors 130, for example turn them on or off receive results directly from the sensor and respond to the results (e.g. repeat measurements or continue measuring responsive to a result). In sonic embodiments of the disclosure, the sensors may be physically connected to the computer 110 or physically independent.

In an exemplary embodiment of the disclosure, computer 110 may include a display 119, a camera 115 and a microphone 117. The display 119 provides pictures and information to the user and the camera 115 and microphone 117 allow the practitioner to see and/or hear the user and/or patient.

In an exemplary embodiment of the disclosure, the computer 110 includes a communication interface 113 for communicating with remote mobile devices 160 over a network 150 (e.g. over the internet, over a cellular network or over a satellite network). Optionally, application 120 displays to the user a list of options including the option to call emergency medical assistance (e.g. 911) or a medical practitioner 175 (e.g. a practitioner on call) to receive assistance in using system 100, for example how to handle a specific situation and/or how to use a specific sensor. Optionally, the practitioner is equipped with a remote mobile device 160, for example a smartphone, tablet or other mobile communication device. In an exemplary embodiment of the disclosure, the remote mobile device 160 includes a display 170 for viewing information transmitted by the computer 110. Optionally, upon receiving notification the medical practitioner 175 connects to computer 110 via a dedicated application 165 or via a standard browser or other standard communication programs, such as SMS, MMS, WhatsApp or similar applications for transmitting text, voice, pictures and/or video data.

In an exemplary embodiment of the disclosure, the medical practitioner 175 is able to view the patient (e.g. via camera 115), hear the patient (e.g. via microphone 117) and/or provide verbal instructions and or execute actions remotely via application 120. For example the practitioner may instruct the local user to deploy a specific sensor on the patient to measure vital signs of the patient. If permitted by the user, the practitioner 175 can view the results and provide further instructions. In some embodiments of the disclosure, the practitioner may set parameters for the sensors and provide instructions to activate the sensor, cease measurements or reactivate. Optionally, the practitioner provides the instructions by clicking on options of his/her mobile device or by providing verbal audio commands. In an exemplary embodiment of the disclosure, the practitioner 175 controls what is displayed to the user, for example to display a specific guide, treatment guideline, open a specific file, send the user a file to display and other options.

In an exemplary embodiment of the disclosure, the verbal commands and/or selection options may include the following;
1. “Display Menu”—To select a selection menu on the display of the local user and/or on the display of the practitioner 175, for example to select a dialing menu, search menu, configuration menu, help menu;
2. “Patient Info”—To select patient information for display, for example to determine the patient's history or sensitivity to treatments;
3. “Display Help”—To Select a the help guide for the local user and/or for the practitioner 175, for example so that the user or practitioner can then select a specific help guide visually explaining how to deploy a sensor;
4. “Select Sensor XXX”—To select a specific sensor 120 for use by the person next to the patient, wherein the sensor can be controlled remotely by the practitioner 175;
5. “Activate Sensor”—To activate a selected sensor 120 and/or display the information delivered by the sensor 120 to the local user and/or remote practitioner 175;
6. “Deactivate Sensor”—To deactivate a selected sensor 120 and/or cease to display the information delivered by the sensor 120 to the local user and/or remote practitioner 175;
7. “Call Help”—To initiate a call from system 100 directly to emergency services, such as 911 to send an ambulance to the location of computer 110 if the patient or user has a problem activating the option on their own. Optionally, the computer may include a GPS 111 to provide the location of the patient;
8. “Activate Application XXX”—To activate a specific service or sub-application of application 120, so that if will be displayed to the local user and/or the remote practitioner.

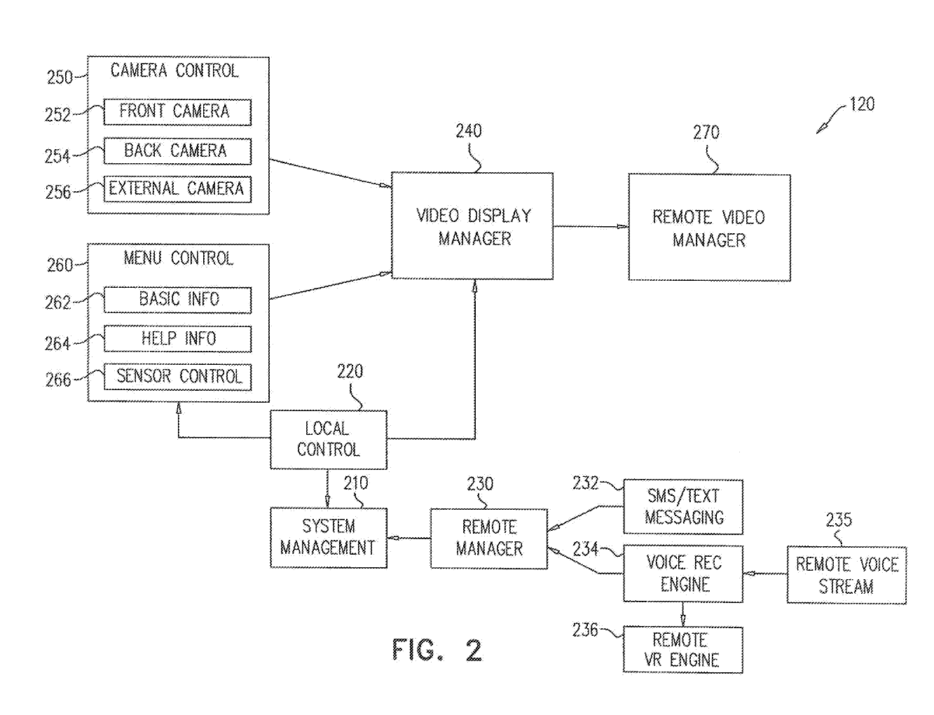

FIG. 2 is a schematic block diagram of elements of application 120 for communicating with a medical practitioner 175, according to an exemplary embodiment of the disclosure. Optionally, application 120 includes a system management routine 210 that is installed on computer 160. In an exemplary embodiment of the disclosure, system management 210 controls a local control 220 to control actions at the local computer 110 and a remote manager 230 to control actions with the remote mobile that is communicating with the application 120. Optionally, local control 220 provides instructions to a video display manager 240 that defines what is displayed locally. Application 120 also includes a camera control 250 that controls reception of images from one or more cameras connected to computer 110, for example a front camera 252, a rear camera 254, an external camera 256 (e.g. USB). The camera control 250 provides images to the video display manager 240 to enable selection of images or videos for display to the user on display 119.

In an exemplary embodiment of the disclosure, application 120 includes a menu control 260 that provides menus for display and use by the user. Optionally, the menus include a basic information menu 262, a help menu 264 and a sensor control menu 266. In an exemplary embodiment of the disclosure, the basic information menu 262 provide details about system 100 and its basic use. The help menu 264 provides the user with help screens explaining how to deal with various medical situations and how to use the elements of system 100. Optionally, the sensor control 266 allows the user to control use of the sensors, set configuration parameters for the sensors and collect results from sensors for transfer to video display manager 240 for display to the user and/or the remote practitioner 175.

In an exemplary embodiment of the disclosure, video display manager 240 receives all displayable information and prepares the information for display. Optionally, some of the information is transferred to a remote video manager process 270 that prepares information and images for transmission to the remote practitioner 175 if permitted by the local user.

In an exemplary embodiment of the disclosure, remote manager 230 receives information from the remote mobile device 160, parses the information and takes appropriate actions based on the requests of the mobile device 160. Optionally, the information may be received by various units such as;
1. A text/SMS messaging unit 232 that receives text information and command from remote mobile device 160;
2. A voice recognition engine 234 that receives voice messages from a remote voice stream 235 and deciphers the voice messages so that application 120 can act on the information. Optionally, the voice recognition engine 234 may optionally send audio information to a remote voice recognition engine 236 to help decipher audio commands.

In an exemplary embodiment of the disclosure, application 120 receives commands from a user locally and from the remote practitioner 175. Optionally, the local user can select which information can be sent to the remote practitioner 175 and which information will be blocked from being sent. Likewise application 120 can be set to allow the practitioner 175 to override selections/actions taken by the local user or allow the local user to override selections and actions taken by the remote practitioner 175.

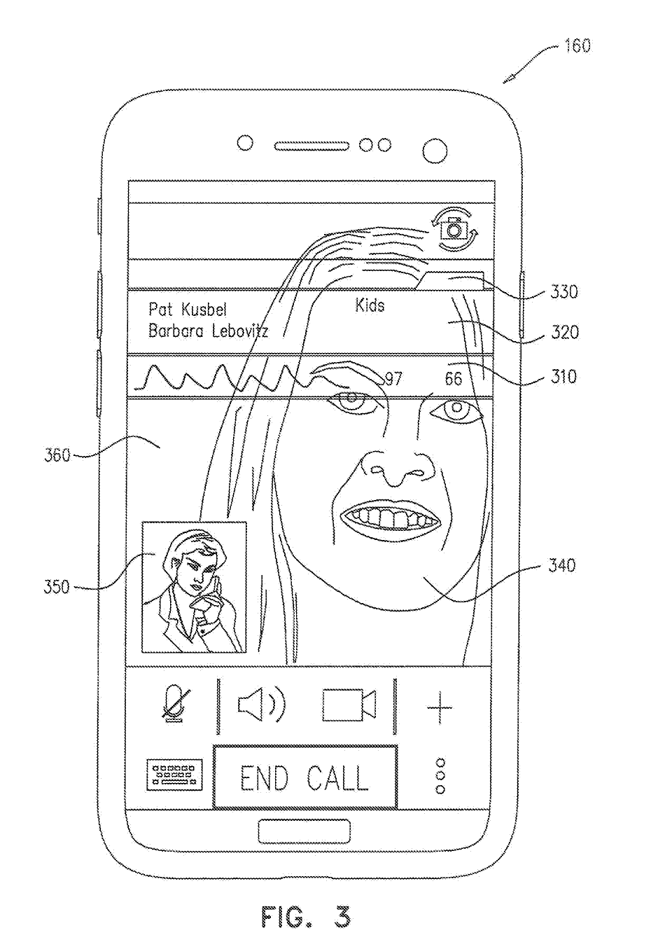

FIG. 3 is a schematic illustration of an exemplary display of remote mobile device 160 for communicating with medical assistance application 120, according to an exemplary embodiment of the disclosure. Optionally, remote mobile device 160 includes a display 360 for displaying information to the practitioner 175. In an exemplary embodiment of the disclosure, mobile device 160 uses dedicated application 165 for communicating with application 120.

Alternatively, application 120 may send mobile device 160 a link to a web that can be executed by a standard web browser.

In an exemplary embodiment of the disclosure, mobile device 160 may display the following information:
1. A live Image or still image 340 from images sent by application 120 (e.g. of the caller by remote video manager 270);
2. One or more tabs 330 to activate command menu's or help menu's that can be selected by the practitioner 175 during the session with application 120.
3. A basic control line 320 providing application information, for example identity of the user that communicated, duration of call and other details;
4. A sensor reading bar 310 that provides sensor readings for the view of the practitioner 175;
5. A practitioner icon 350 showing a symbol of the practitioner that is displayed to the user on computer 110.

In an exemplary embodiment of the disclosure, application 120 can be stored on a non-transitory memory device and be delivered to and installed on a computerized device to perform the functions described above.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled In the art that the present invention is not limited to what has been particularly shown and described hereinabove. Rather the scope of the present invention is defined only by the claims, which follow.

I/We claim:

1. A system for treating a patient, comprising:
a computerized device comprising a display and a communication interface for communicating-with remote mobile devices;
one or more sensors for measuring medical parameters of a user; wherein the sensors are controlled by the computerized device and transfer recorded measurements to the computerized device;
an application executed on the computerized device; wherein the application allows a remote practitioner with a remote mobile device to communicate with the computerized device to control the sensors.

2. The system according to claim 1, wherein the application accepts verbal audio commands from the remote practitioner to control the sensors.

3. The system according to claim 1, wherein the application accepts text commands from the remote practitioner to control the sensors.

4. The system according to claim 1, wherein at least one sensor communicates with the computerized device via a USB connection.

5. The system according to claim 1, wherein at least one sensor communicates with the computerized device via a direct local wireless connection.

6. The system according to claim 1, wherein the application allows the remote practitioner to control the display of menus and help guides on the display of the computerized device.

7. The system according to claim 1, wherein the application allows the remote practitioner to provide verbal messages that are displayed as text on the display of the computerized device.

8. The system according to claim 1, wherein the application accepts images from a camera and transmits them to the remote practitioner to view.

9. The system according to claim 1, wherein the one or more sensors are selected from the group consisting of a pulse oximeter, a blood pressure sensor, a stethoscope, an otoscope and a thermometer.

10. The system according to claim 1, wherein the application transmits the readings of the sensors to the remote practitioner to view.

11. The system according to claim 1, wherein the computerized device is a tablet.

12. The system according to claim 1, wherein the application allows a remote practitioner to initialize a call for help from emergency services by the computerized device.

13. A method of treating a patient by a medical practitioner from a remote location, comprising:
activating a computerized device comprising a display and a communication interface for communicating with remote mobile devices;
deploying one or more sensors for measuring medical parameters of a user; wherein the sensors communicate and receive instructions from the computerized device;
initializing an application executed on the computerized device;
forming communication between the application and a remote mobile device used by a remote practitioner;
enabling the remote practitioner to control the sensors remotely by the computerized device.

14. The method according to claim 13, wherein the application accepts verbal audio commands from the remote practitioner to control the sensors.

15. The method according to claim 13, wherein the application accepts text commands from the remote practitioner to control the sensors.

16. The method according to claim 13, wherein the application allows the remote practitioner to control the display of menus and help guides on the display of the computerized device.

17. The method according to claim 13, wherein the application allows the remote practitioner to provide verbal audio messages that are displayed as text on the display of the computerized device.

18. The method according to claim 13, wherein the application accepts images from a camera and transmits them to the remote practitioner to view.

19. The method according to claim 13, wherein the application transmits the readings of the sensors to the remote practitioner to view.

20. A non-transitory memory device storing an executable program that can be executed on a computerized device to perform a method as set forth in claim 13.
